(19) United States
(12) Patent Application Publication
(10) Pub. No.: US 2022/0221445 A1
(43) Pub. Date: Jul. 14, 2022

(54) USES OF PATIENT-DERIVED INTESTINAL ORGANOIDS FOR CELIAC DISEASE DIAGNOSIS SCREENING AND TREATMENT

(71) Applicant: The Board of TrusteesoftheLelandStanfordJuniorUniversity, Stanford, CA (US)

(72) Inventors: Calvin J. Kuo, Palo Alto, CA (US); Antonio Santos, Palo Alto, CA (US)

(21) Appl. No.: 17/613,430

(22) PCT Filed: Jun. 3, 2020
(86) PCT No.: PCT/US2020/035964

§ 371 Date: Nov. 22, 2021

Related U.S. Application Data
(60) Provisional application No. 62/856,481, filed on Jun. 3, 2019.

(51) Int. Cl. G01N 33/50; C12N 5/0783; C12N 5/071; G01N 33/564

(52) U.S. Cl. CPC G01N 33/5082; C12N 5/0636; C12N 5/0679; C12N 2503/04; G01N 33/564; G01N 2800/065; C12N 5/0697

(57) ABSTRACT

Air-liquid interface organoid cultures are initiated from human small intestine biopsy tissue comprising both the synintestinal epithelium and native intestinal immune cells, without reconstitution, which may be obtained from an individual pre-disposed or suffering from celiac disease. The organoid cultures exhibit T cell activation in response to in vitro gluten challenge and provide tools for a novel diagnostic method for celiac disease. Diagnosis may comprise the addition of immunogenic gluten-derived peptides into the organoid cultures, and assessing hallmarks of active celiac disease, including without limitation: 1) gliadin-presentation, resulting T-cell responses, such as 2) expansion and 3) activation, 4) epithelial-cell death and consequent 5) increased proliferative epithelial cell responses to gliadin. Celiac patients, either in GRD or GFD, test positive for these tests. In other embodiments the organoids are used to test responses to candidate therapeutic agents, assessing reduction of gliadin-dependent (1) T cell activation or expansion, or (2) organoid epithelial cell death.

### CROSS REFERENCE

This application claims benefit and is a 371 of PCT Application No. PCT/US2020/035964, filed Jun. 3, 2020, which claims the benefit of U.S. Provisional Patent Application No. 62/856,481, filed Jun. 3, 2019, which applications are incorporated herein by reference in their entirety.

### BACKGROUND

The pathological reaction to gluten occurs in genetically predisposed individuals, generally celiac patients are either HLA-DQ2⁺ (90%) or HLA-DQ8⁺, However, expression of these MHC II haplotypes per se is not sufficient to develop the disease, indicating that other factor(s) are necessary to trigger the development of celiac sprue.

Diagnosis of celiac disease (CeD) usually starts by testing for Transglutaminase 2 (TG2) autoantibody in the patient's serum, which if positive is followed by an endoscopy with intestinal biopsies necessary for histological analysis—a scalloped appearance of the duodenal mucosa caused by villi blunting confirms the CeD diagnosis.

However, individuals often start a gluten-free diet (GFD) on their own prior to their gastroenterology consultation, thus testing negative for TG2 serum autoantibodies and presenting a normal-looking duodenal mucosa in the initial diagnostic tests, although genetically testing positive for HLA-DQ2 or HLA-DQ8. Those suspected-celiac patients have therefore to go on a gluten rich-diet (GRD) for 4 to 6 weeks to get a definite diagnosis. In positive cases, individuals experience distressful symptoms such as abdominal pain, diarrhea, bloating and fatigue throughout this fastidious process before serologic and histologic analyses are repeated and a diagnosis is achieved. On the other hand, other suspected celiac-patients in a gluten-containing diet present a normal duodenal mucosa despite having positive serologic tests and being DQ2+ or DQ8+. Whether or not to introduce a GFD diet in these cases is a controversial issue as it is unclear what proportion of these individuals develops clinically significant mucosal injury.

Current methods to diagnose celiac disease rely on genetic and serological tests and histological analysis of duodenal biopsies, or requiring the patient to ingest gluten, incurring symptoms (so called “oral gluten challenge”), Although these methods are efficient in some cases of celiac-patients in a gluten-rich diet (GRD), they still require 3 completely different analytical approaches that take several weeks to complete and significant labor. In addition, for suspected celiac individuals already in a gluten-free diet (GFD), these tests do not allow for a conclusive diagnosis, leading to the need for a return to a 4-6 week-long GRD, with severe painful symptoms for individuals that are indeed celiac. The currently used diagnostic tools are also not conclusive for some celiac patients that, although are in a GRD, do not develop any enteropathy. The gliadin challenge in small intestine organoid cultures allows for a less-invasive simpler diagnosis (no blood collection or genetic testing required) that from biopsy collection to diagnosis takes only approximately 15 to 20 days to complete.

Noninvasive and faster diagnostic methods for celiac disease are of great interest. Further, in vitro systems to test personalized responses to celiac disease therapies are also urgently needed.

### SUMMARY

Compositions and methods are provided for diagnosis of celiac disease and screening of candidate agents for treatment of celiac disease. The methods described herein utilize air-liquid interface organoid in vitro cultures derived from tissue of the small intestine, where the cultures comprise epithelial cells and immune stroma tissue from the small intestine, for example epithelial cells and immune stroma cultured from a small intestine tissue sample. In some embodiments the sample is human small intestine biopsy tissue comprising both syngeneic intestinal epithelium and native intestinal immune cells, providing for both sets of cells in the culture from a single sample, and without reconstitution. The tissue is cultured in a medium that supports maintenance and activity of both epithelial and immune cells. The tissue sample may be obtained from an individual suspected of having celiac disease, or an individual suspected of a pre-disposition to pre-disposed to celiac disease; or a normal control. In some embodiments a culture comprises exogenously supplied gluten-derived peptides in a dose effective to activate immune cells present in the culture.

The in vitro cultured cells provide tools for a novel diagnostic method for celiac disease. The diagnostic methods can comprise a method of: adding gluten-derived peptides into the organoid cultures in a dose effective to activate immune cells present in the culture, and assessing the culture for the development of hallmarks of active celiac disease, which hallmarks may include, without limitation: 1) gliadin-presentation by immune cells that results in T-cell responses, such as 2) T cell expansion and 3) T cell activation; 4) epithelial-cell death and consequent 5) increased proliferative epithelial cell responses to gliadin. It is shown herein that celiac patients, either in GRD or GFD, test positive for these tests. In other embodiments the organoids are used to test responses of candidate therapeutic agents, assessing reduction of gliadin-dependent (1) T cell activation or expansion, or (2) organoid epithelial cell death.

Air-liquid interface (ALI) organoids have both epithelial and stromal components from organ tissue used to initiate the culture, including human small intestinal tissue. The ALI method allows culturing epithelium and stroma together as a cohesive 3-dimensional unit that recapitulates the function and the micro-anatomy of the organ of origin. In ALI, adequate oxygenation is achieved by culturing microscopic fragments of tissue embedded in a collagen matrix within a trans-well (“inner dish”) in which direct air exposure is obtained from the top; whilst contact with tissue culture media contained in an “outer dish”; is obtained from the bottom via the trans-well permeable membrane.

Using the ALI method, cultured human small intestine organoids from active celiac patients (celiacs in GRD), remission patients (celiacs in GFD) and healthy controls have been successfully initiated using 1, 2, 3, 4, 5, 6, 7, 8 or more, e.g. from 4 to 8, small biopsy pieces. These organoids allow measuring T-cell expansion and activation through, for example, RT-qPCR of specific transcripts, such as IFN-gamma (IFNG), Perforin 1 (PRF1) and Granzyme B (GZMB) after T-cell staining and isolation using FACS (Fluorescence Activated Cell Sorting); and epithelial-injury responses, for example such as cell-death through apoptotic markers (e.g. Annexin V or cleaved-caspase 3) and proliferation markers (e.g. Ki67), using FACS and immunofluorescence microscopy. There is excellent correlation between the organoid immune response to gliadin (gluten challenge) and patient clinical status, where organoids from active celiac patients mount an immune response to gliadin, while organoids from patients without celiac disease do not. A diagnosis of celiac disease or predisposition to celiac disease may be made when there is at least one disease-associated response after gluten challenge, and there may be two, three or all disease-associated responses after gluten challenge.

In another aspect of the invention, a method is provided for in vitro screening for agents for their effect on cells of different tissues, including processes of celiac disease initiation and treatment, and including the use of experimentally modified cultures described above. Tissue explants cultured by the methods described herein are exposed to candidate agents. Agents of interest include pharmaceutical agents, e.g. small molecules, antibodies, peptides, etc., and genetic agents, e.g. antisense, RNAi, expressible coding sequences, and the like, e.g. expressible coding sequences for candidate secreted growth factors, cytokines, receptors or inhibitors thereof, or other proteins of interest, and the like. In some embodiments the effect of candidate therapeutic agents on celiac disease-related immune responses or their downstream effects on apoptosis or growth of intestinal epithelial cells or stem cells is determined, for example where agents may include, without limitation, chemotherapy, monoclonal antibodies or other protein-based agents, radiation/radiation sensitizers, cDNA, siRNA, shRNA, small molecules, and the like. Effects on immune responses can be detected by measuring the prevalence of different types of immune cells, their expression of activation markers, gene expression, proteome, or expression of celiac disease-relevant T cell receptor sequences, for instance. In other embodiments, the effects of such candidate therapeutics on stem cells is determined. Agents active on tissue-specific stem cells are detected by change in growth of the tissue explants and by the presence of multilineage differentiation markers indicative of the tissue-specific stem cell. In addition, active agents are detected by analyzing tissue explants for long-term reconstitutive activity. Methods are also provided for using the explant culture to screen for agents that modulate tissue function.

Methods are provided for screening cells in a population, e.g. a complex population of multiple cells types, a population of purified cells isolated from a complex population by sorting, culture, etc., and the like, for the ability to regulate gluten-dependent changes in immune cells, differentiated intestinal epithelial cells or intestinal stem cells within the culture. This method entails co-culture of the aforementioned detectably labeled candidate cells with the tissue explant of the invention to assay modulation of celiac disease-related endpoints. This could include added immune cells that suppress or promote the gluten-dependent immune responses within the organoids. Candidate cells with stem cell potential are detected by an increase in growth of the cultured explant above basal levels despite gluten treatment and colocalization of multilineage differentiation markers indicative of the presence of tissue-specific stem cells with the labeled candidate cells.

In another aspect of the invention, a method is provided for in vitro screening of agents for cytotoxicity to different tissues, by screening for toxicity to explant cultures of the invention. In yet another embodiment, a method is provided to assess drug absorption by different tissues, by assessing absorption of a drug by explant cultures of the invention.

### BRIEF DESCRIPTION OF THE DRAWINGS

FIG. 1A) Top left side- Human small intestine biopsies are minced into microscopic fragments and embedded in a collagen matrix within a trans-well (“inner dish”) in which direct air exposure is obtained from the top; whilst contact with tissue culture media contained in an “outer dish” is obtained from the bottom via the trans-well permeable membrane, generating an Air-Liquid Interface (ALI). Top right-side- Hematoxylin and Eosin (H&E) staining of a variety of human small intestine organoids grown for 12 to 14 days, showing the formation of a lumen in the middle surrounded by a mucosal layer often showing villus-like structures. Bottom left-side-Hematoxylin and Eosin (H&E) staining of the villi structures (top) and immunofluorescence microscopy of the proliferation marker Ki-67 (green) and DAPI (blue) indicating that cell division in the organoids occurs predominantly in the bottom cell layers (arrows). Bottom right-side- Immunofluorescence microscopy of the epithelial cell marker E-cadherin (white), of the T-cell marker CD3 (green) and of the macrophage marker CD14 (red), showing that these intestinal organoids contain an immune stroma. FIG. 1B) Single cell RNA-seq transcriptional profiling of the cells from the organoids that are relevant for celiac disease, which includes immune cells (CD45⁺), in particular CD4⁺ and CD8+ T-cells, CD79a⁺ B cells and EpCam⁺ epithelial cells. (FIG. 1C) Scheme of the methodology used to model celiac disease using ALI small intestine human organoids. 4-8 biopsy bites are collected and minced into microscopic fragments that are grown from 7-12 days and then treated with either a control peptide (CLIP) or GLIADIN. The organoids are harvested from the culture 2 days later for analysis.

FIG. 2A) Single cells harvested from the organoids were sorted and analyzed after FACS immunostaining for the epithelial cell marker EpCam and FIG. 2B) the apoptosis marker Annexin V together with a dead cell marker AmCyan, showing that treatment with gliadin but not CLIP leads to an increase in cell death (top-right quadrant) only in celiac organoids and not in healthy-organoids. FIG. 2C) Depletion of T-cells with a CD3 antibody given for 2 days prior to the in vitro gliadin or CLIP treatments abrogated the increase in cell death induced by gliadin in celiac-organoids. FIG. 2D) Immunofluorescence microscopy of the apoptosis marker cleaved caspase-3 (green) and the epithelial cell marker E-cadherin (red) confirmed an increase in epithelial cell death in celiac-organoids treated with gliadin but no effects in healthy-organoids. DAPI (blue).

FIG. 3A) Single cells harvested from the organoids were sorted by FACS and analyzed after staining to exclude dead cells and immunostaining for EpCam to isolate the epithelial cells. FIG. 3B) Sorted epithelial cells were processed for gene expression analysis by RT-qPCR (Real-Time quantitative PCR), which revealed an increase in transcripts upon gliadin treatment versus CLIP treatment of the epithelial stem cell marker LGR5 and the proliferative markers FIG. 3C) CCND1 and FIG. 3D) PCNA, in organoids derived from active celiac patients in a gluten-rich diet (GRD) and in organoids derived from remission celiac patients in a gluten-free diet (GFD) but not in non-celiac healthy-derived organoids. FIG. 3E) Immunofluorescence microscopy of the proliferation marker Ki67 (green) and the epithelial cell marker E-cadherin (red) confirmed an increase in epithelial cell proliferation in celiac organoids treated with gliadin but no changes when celiac organoids were treated with CLIP. DAPI (blue). Organoids were placed into a media to induce intestinal epithelial differentiation and induce quiescence prior to the in vitro gliadin treatment. FIG. 3F) Quantification of the fold-increase in number of Ki67⁺ cells treated with gliadin versus CLIP revealed a 6× fold increase in proliferative cells in organoids derived from active celiac patients.

FIG. 4A) Single cells harvested from the organoids were sorted and analyzed after FACS staining to exclude dead cells and immunostaining for CD45, CD3, CD4 or CD8. FIG. 4B) The number of CD45⁺, CD3⁺ T cells was calculated as a fold-increase comparing gliadin to CLIP treatments, revealing an increase in T cells only in organoids derived from active celiac patients, but no significant changes were observed in remission or healthy organoids. FIG.4C) The quantification of CD45⁺, CD3⁺, CD4⁺ T helper cells and of the FIG. 4D) CD45⁺, CD3⁺, CD8⁺ cytotoxic T cells, showed an expansion of these two subtypes of T cells only in organoids derived from active celiac patients, but no significant changes were observed in remission or healthy organoids. FIG. 4E) Immunofluorescence microscopy of CD3⁺ T cells and the epithelial cell marker E-cadherin (white) confirmed the expansion of T-cells observed by FACS in celiac-organoids treated with gliadin but no changes when celiac-organoids were treated with CLIP. DAPI (blue).

FIG. 5A) Single cells harvested from the organoids were sorted by FACS to exclude dead cells followed by immunostaining for CD45, CD3 and CD4 to isolate T helper cells that were then processed for gene expression analysis by RT-qPCR. This revealed an increase in transcripts upon 48 h gliadin treatment versus CLIP treatment of several pro-inflammatory cytokines such as IL-2, IL-22, IL-17A, IL-10, IL-25 and IFNG; of CD38, a memory T-cell marker and CD25, the IL-2 receptor; and of proliferation markers such as CCND1 and PCNA in organoids derived from active celiac patients and to some extent in organoids derived from remission celiac patients, but not in non-celiac healthy-derived organoids. FIG. 5B) Single cells harvested from the organoids were sorted and analyzed after staining to exclude dead cells and immuno-staining for CD45, CD3, CD8 to isolate cytotoxic T-cells. that were then processed for gene expression analysis by RT-qPCR, which revealed an increase in transcripts upon 48 h gliadin versus CLIP treatments of several activation markers such as IFNG, PRF1 and GZMB; of CD38, a memory T-cell marker and CD25, the IL-2 receptor; and of proliferation markers such as CCND1 and PCNA, in organoids derived from active celiac patients and to some extent in organoids derived from remission celiac patients but not in non-celiac healthy-derived organoids.

### DEFINITIONS

In the description that follows, a number of terms conventionally used in the field of cell culture are utilized extensively. In order to provide a clear and consistent understanding of the specification and claims, and the scope to be given to such terms, the following definitions are provided.

The term “cell culture” or “culture” means the maintenance of cells in an artificial, in vitro environment. It is to be understood, however, that the term “cell culture” is a generic term and may be used to encompass the cultivation not only of individual cells, but also of tissues or organs.

The term “culture system” is used herein to refer to the culture conditions in which the subject explants are grown that promote prolonged tissue expansion with proliferation, multilineage differentiation and recapitulation of cellular and tissue ultrastructure.

“Gel substrate”, as used herein has the conventional meaning of a semi-solid extracellular matrix. Gel described here in includes without limitations, collagen gel, matrigel, extracellular matrix proteins, fibronectin, collagen in various combinations with one or more of laminin, entactin (nidogen), fibronectin, and heparin sulfate; human placental extracellular matrix.

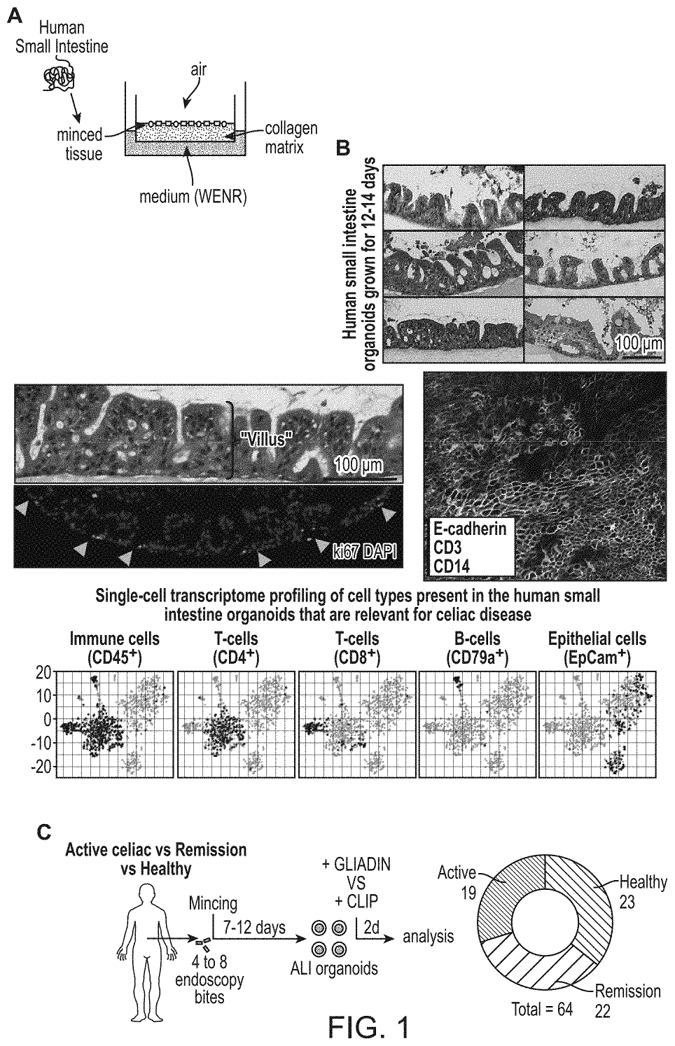

An “air-liquid interface” is the interface to which the intestinal cells are exposed to in the cultures described herein. The primary tissue may be mixed with a gel solution which is then poured over a layer of gel formed in a container with a lower semi-permeable support, e.g. a membrane. This container is placed in an outer container that contains the medium such that the gel containing the tissue in not submerged in the medium. The primary tissue is exposed to air from the top and to liquid medium from the bottom (FIG. 1A).

By “container” is meant a glass, plastic, or metal vessel that can provide an aseptic environment for culturing cells.

The term “explant” is used herein to mean a piece of tissue and the cells thereof originating from mammalian tissue that is cultured in vitro, for example according to the methods of the invention. The mammalian tissue from which the explant is derived may obtained from an individual, i.e. a primary explant, or it may be obtained in vitro, e.g. by differentiation of induced pluripotent stem cells.

The term “organoid” is used herein to mean a 3-dimensional growth of mammalian cells in culture that retains characteristics of the tissue in vivo, e.g. prolonged tissue expansion with proliferation, multilineage differentiation, recapitulation of cellular and tissue ultrastructure, etc. A primary organoid is an organoid that is cultured from an explant, i.e. a cultured explant. A secondary organoid is an organoid that is cultured from a subset of cells of a primary organoid, i.e. the primary organoid is fragmented, e.g. by mechanical or chemical means, and the fragments are replated and cultured. A tertiary organoid is an organoid that is cultured from a secondary organoid, etc.

The phrase “mammalian cells” means cells originating from mammalian tissue. Typically, in the methods of the invention pieces of tissue are obtained surgically and minced to a size less than about 1 mm³, and may be less than about 0.5 mm³, or less than about 0.1 mm³. “Mammalian” used herein includes human, equine, bovine, porcine, canine, feline, rodent, e.g. mice, rats, hamster, primate, etc. “Mammalian tissue cells” and “primary cells” have been used interchangeably.

“Tissue-specific stem cells” is used herein to refer to multipotent stem cells that reside in a particular tissue and are capable of clonal regeneration of cells of the tissue in which they reside, for example the ability of hematopoietic stem cells to reconstitute all hematopoietic lineages, or the ability of neuronal stem cells to reconstitute all neuronal/glial lineages. “Progenitor cells” differ from tissue-specific stem cells in that they typically do not have the extensive self-renewal capacity, and often can only regenerate a subset of the lineages in the tissue from which they derive, for example only lymphoid or erythroid lineages in a hematopoietic setting, or only neurons or glia in the nervous system.

Culture conditions of interest provide an environment permissive for differentiation, in which the complex cell system from an explant cells will proliferate, differentiate, or mature in vitro. Such conditions may also be referred to as “differentiative conditions”. Features of the environment include the medium in which the cells are cultured, any growth factors or differentiation-inducing factors that may be present, and a supporting structure (such as a substrate on a solid surface) if present.

The term “multi-lineage differentiation markers” means differentiation markers characteristic of different cell-types. These differentiation markers can be detected by using an affinity reagent, e.g. antibody specific to the marker, by using chemicals that specifically stain a cell type, etc as known in the art.

“Ultrastructure” refers to the three-dimensional structure of a cell or tissue observed in vivo. For example, the ultrastructure of a cell may be its polarity or its morphology in vivo, while the ultrastructure of a tissue would be the arrangement of different cell types relative to one another within a tissue.

The term “candidate cells” refers to any type of cell that can be placed in co-culture with the tissue explants described herein. Candidate cells include without limitations, mixed cell populations, ES cells and progeny thereof, e.g. embryoid bodies, embryoid-like bodies, embryonic germ cells.

The term “candidate agent” means any oligonucleotide, polynucleotide, siRNA, shRNA, gene, gene product, peptide, antibody, small molecule or pharmacological compound that is introduced to an explant culture and the cells thereof as described herein to assay for its effect on the explants.

The term “contacting” refers to the placing of candidate cells or candidate agents into the explant culture as described herein. Contacting also encompasses co-culture of candidate cells with tissue explants for at least 1 hour, or more than 2 hrs or more than 4 hrs in culture medium prior to placing the tissue explants in a semi-permeable substrate. Alternatively, contacting refers to injection of candidate cells into the explant, e.g. into the lumen of an explant.

“Screening” refers to the process of either co-culturing candidate cells with or adding candidate agents to the explant culture described herein and assessing the effect of the candidate cells or candidate agents on the explant. The effect may be assessed by assessing any convenient parameter, e.g. the growth rate of the explant, the presence of multilineage differentiation markers indicative of stem cells, etc.

Gluten-derived peptides. In some embodiments a culture described herein comprises exogenously supplied gluten-derived peptides in a dose effective to activate immune cells present in the culture. Various gluten peptides are known in the art to be immunogenic, e.g. as described, inter alia, in U.S. Pat. No. 7,462,688; Sjostrom, H., et al. Scand J Immunol 48, 111-115 (1998); Dorum S. et al., J. Proteome Res. 2009; 8:1748-55; Mothes Adv Clin Chem 2007; 44:35-63; each herein specifically incorporated by reference. A major component of gluten is the protein gliadin. Gliadin peptides derived from Triticum aestivum (wheat) are the main immunotoxic antigens present in celiac disease. They are substrates for tissue transglutaminase, which specifically deamidates glutamine residues within these peptides, and therefore strongly increases their immunogenicity. It has been found that a-gliadin derived epitopes that are frequently recognized by patient T cells showed a significant higher level of deamidation compared to the majority of epitopes from γ-gliadin that are less frequently recognized. The degree of deamidation of individual residues within a peptide also seems to influence whether some epitopes are better recognized in context of DQ2 or DQ8.

Gliadin peptides for this purpose may be from about 6 to 35 amino acids in length, including for example 33-mers, 26-mers, 14-mers, 13-mers, etc. and are optionally deaminidated. Peptides for this purpose are commercially available, e.g. gliadin-α1 (14 aa, Genscript) and gliadin-α2 (13 aa, Genscript). The gluten-derived peptides may be provided in the culture at a concentration of from about 0.5 μM to about 100 μM, usually from about 1 μM, about 5 μM, about 10 μM to about 100, about 50 about 25 μM.

Culture systems and methods are provided. By long term culture, it is meant continuous growth of the explant for extended periods of time, e.g. for 15 days or more, for 1 month or more, for 2 months or more, for 3 months or more, for 6 months or more, or up to a year, or more. By continuous growth, it is meant sustained viability, organization, and functionality of the tissue. For example, unless experimentally modified, proliferating cells in a tissue explant that undergoes continuous growth in the culture systems of the present application will continue to proliferate at their natural rate, while non-proliferative, e.g. differentiated, cells in the tissue explant will remain in a quiescent state. Because of this, explants cultured by the subject methods are referred to as “organoids”.

Explants cultured in this way may be sustained for a long term at physiological temperatures, e.g. 37° C., in a humidified atmosphere of, e.g. 5% CO₂ in air. Medium is changed about every 10 days or less, e.g. about 1, 2, or 3 days, sometimes 4, 5, or 6 days, in some instances 7, 8, 9, 10, 11 or 12 days, usually as convenient.

For the purposes of the present invention, explants are often cultured from about 5 days, about 6 days, about 7 days, about 8 days, about 10 days, about 12 days, about 15 days, and may be cultured for not more than about 30 days. The immune responses may be observed within about 1 day following gluten challenge, within about 2 days, within about 3 days, within about 4 days, within about 5 days, within about 1 to about 2 weeks.

In some embodiments, tissue, i.e. primary tissue, is obtained from a mammalian organ. The tissue may be from any mammalian species, e.g. human, equine, bovine, porcine, canine, feline, rodent, e.g. mice, rats, hamster, primate, etc. The mammal may be of any age, e.g. a fetus, neonate, juvenile, adult.

Tissue may be obtained by any convenient method, e.g. by biopsy, e.g. during endoscopy, during surgery, by needle, etc., and is typically obtained as aseptically as possible. Upon removal, tissue is immersed in ice-cold buffered solution, e.g. PBS, Ham's F12, MEM, culture medium, etc. Pieces of tissue are minced to a size less than about 1 mm³, and may be less than about 0.5 mm³, or less than about 0.1 mm³. The minced tissue is mixed with a gel substrate, e.g. a collagen gel solution, e.g. Cellmatrix type I-A collagen (Nitta Gelatin Inc.); a matrigel solution, etc. Subsequently, the tissue-containing gel substrate is layered over a layer of gel (a “foundation layer”) in a container with a lower semi-permeable support, e.g. a membrane, supporting the foundation gel layer, and the tissue-containing gel substrate is allowed to solidify. This container is placed into an outer container containing a suitable medium, for example HAMs F-12 medium supplemented with fetal calf serum (FCS) at a concentration of from about 1 to about 25%, usually from about 5 to about 20%, etc.

The arrangement described above allows nutrients to travel from the bottom, through the membrane and the foundation gel layer to the gel layer containing the tissue. The level of the medium is maintained such that the top part of the gel, i.e. the gel layer containing the explants, is not submerged in liquid but is exposed to air. Thus the tissue is grown in a gel with an air-liquid interface. A description of an example of an air-liquid interface culture system is provided in Ootani et al. in Nat Med. 2009 June; 15(6):701-6, the disclosure of which is incorporated herein in its entirety by reference.

The continued growth of explants may be confirmed by any convenient method, e.g. phase contrast microscopy, stereomicroscopy, histology, immunohistochemistry, electron microscopy, etc. In some instances, cellular ultrastructure and multi-lineage differentiation may be assessed. Ultrastructure of the intestinal explants in culture can be determined by performing Hematoxylin-eosin staining, PCNA staining, electron microscopy, and the like using methods known in the art. Multi-lineage differentiation can be determined by performing labeling with antibodies to terminal differentiation markers, e.g. as described in greater detail below. Antibodies to detect differentiation markers are commercially available from a number of sources.

In some embodiments, the growth of the explants in culture may be stimulated by introducing R-spondin into the culture medium. R-spondin1 (Rspo1, Genbank Accession NP_001033722) is a secreted glycoprotein which synergizes with Wnt to activate β-catenin dependent signaling (Kim et al., 2005, Kim et al., 2006). Explants cultured by the subject methods that are exposed to RSpo1 exhibit increased growth (see Ootani et al. in Nat Med. 2009 June; 15(6):701-6). The factors may be added to the culture at a concentration of from about 500 ng/ml, at least about 0.5 μg/ml, at least about 50 μg/ml and not more than about 1 mg/ml, with change of medium every 1-2 days.

In some embodiments EGF is provided in the culture medium, for example at a concentration of from about of at least about 1 ng/ml, at least about 10 ng/ml, at least about 50 ng/ml and not more than about 1 mg/ml.

In some embodiments, noggin is provided in the culture medium, for example at a concentration of from about of at least about 1 ng/ml, at least about 10 ng/ml, at least about 50 ng/ml and not more than about 1 mg/ml.

In some embodiments the medium comprises an activator of the WNT pathway, which can include but are not limited to, e.g., CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile), WNT family ligands (e.g., including but not limited to Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b, etc.), RSPO co-agonists (e.g., RSPO2), lithium chloride, TDZD8 (4-Benzyl-2-methyl-1,2,4-thiadiazolidine-3,5-dione), BIO-Acetoxime ((2′Z,3′E)-6-Bromoindirubin-3′-acetoxime), A1070722 (1-(7-Methoxyquinolin-4-yl)-3-[6-(trifluoromethyl)pyridin-2-yl]urea), HLY78 (4-Ethyl-5,6-Dihydro-5-methyl-[1,3]dioxolo[4,5-j]phenanthridine), CID 11210285 hydrochloride (2-Amino-4-(3,4-(methylenedioxy)benzylamino)-6-(3-methoxyphenyl)pyrimidine hydrochloride), WAY-316606, (hetero)arylpyrimidines, IQ1, QS11, SB-216763, DCA, and the like. The WNT activator can be provided at a concentration of from about 0.5 μM, 1 μM, 5 μM, 10 μM, up to about 1 mM.

In some embodiments a DMEM based media is supplemented with each of R-spondin, a WNT agonist, noggin, and EGF. The medium may optionally further comprise, for example, inhibitors of p160ROCK, and of p38 MAP kinase, for example Y-27632, which is a biochemical tool used in the study of the rho-associated protein kinase (ROCK) signaling pathways and SB 202190, each at a concentration of from about 0.5 μM, 1 μM, 5 μM, 10 μM, up to about 1 mM.

In some embodiments, the cells in the cultured explants are experimentally modified. For example, the explant cells may be modified by exposure to viral or bacterial pathogens, e.g. to develop a reagent for experiments to assess the anti-viral or anti-bacterial effects of therapeutic agents. The explant cells may be modified by altering patterns of gene expression, e.g. by providing reprogramming factors to induce pluripotency or otherwise alter differentiation potential, or to determine the effect of a gain or loss of gene activity on the ability of cells to form an explant culture or on the ability of cells to undergo tumor transformation. The explant cells may be modified such that they are transformed with growth factors or cytokines or other genes to modulate celiac disease phenotypes on immune cells or intestinal epithelial cells.

Experimental modifications may be made by any method known in the art, for example, as described below with regard to methods for providing candidate agents that are nucleic acids, polypeptides, small molecules, viruses, etc. to explants and the cells thereof for screening purposes.

### Diagnostic Methods

Compositions and methods are provided for diagnosis of celiac disease. The methods utilize air-liquid interface organoid in vitro cultures derived from tissue of the small intestine, where the cultures comprise epithelial cells and immune stroma tissue from the small intestine Usually the sample is human small intestine biopsy tissue comprising both syngeneic intestinal epithelium and native intestinal immune cells, providing for both sets of cells in the culture from a single sample, and without reconstitution. The tissue sample may be obtained from an individual suspected of having celiac disease, or an individual suspected of a pre-disposition to pre-disposed to celiac disease; or a normal control.

Following establishment of the organoid culture in vitro, e.g. after about 5 to about 14 days in culture, the culture is provided with a gluten challenge by adding to the medium exogenously supplied gluten-derived peptides in a dose effective to activate immune cells present in the culture. The response to the gluten challenge is assessed from about 1 day to about 5 days, e.g. 1, 2, 3, 4, 5, days or more following the challenge. The response to challenge can be evidenced by an increase in one or more of the hallmarks described below as indicative of a celiac disease phenotype, and may be evidenced by an increase in 1, 2, 4, 5 hallmarks. An increase is evidenced as at least a 5%, 10%, 25%, 50% or more increase relative to the response of a normal control, either a control non-gluten peptide, or in reference to a normal organoid not predisposed to celiac disease.

Hallmarks may include, without limitation: 1) gliadin-presentation by immune cells that results in T-cell responses, such as 2) T cell expansion and 3) T cell activation; 4) epithelial-cell death and consequent 5) increased proliferative epithelial cell responses to gliadin. It is shown herein that celiac patients, either in GRD or GFD, test positive for one or more of these tests.

Celiac disease is characterized by seminal histologic characteristics, including the well-known villus blunting, resulting from immune-mediated epithelial cell death. Less appreciated in CeD is the uniform and simultaneous presence of crypt hypertrophy, which may represent compensatory proliferation. For one hallmark, when active CeD cultures are treated with gliadin peptides as a gluten challenge, compared to a negative control peptide (CLIP), the gluten challenge induces epithelial cell death in ALI organoids from patients with CeD, which can be active or in remission. In another hallmark, the gluten challenge also increases expression of proliferative markers in epithelial cells from patients with CeD, which can be active or in remission. Measurement of cell death may be quantified, for example, by staining cells in the culture for apoptotic markers, e.g. Annexin V, cleaved-caspase 3, etc. Measurement of proliferation may be quantified, for example, by staining cells in the culture of proliferation markers, e.g. Ki67, CCND1, PCNA, etc. Flow cytometry, immunofluorescence microscopy, mass cytometry, etc. can be used to detect the level of staining. Optionally the culture is counterstained with EpCam to gate on epithelial cells.

Other hallmarks of disease following gluten challenge include T cell responses. Following a gluten challenge is disease predisposed organoids, there is an increase in CD3⁺, CD4⁺ and CD8⁺ T cells , although the T cell proliferation may be specific for active CeD organoids and not for organoids from control/non-CeD or remission celiac (i.e., asymptomatic CeD patients on a GFD). Measuring T-cell expansion and activation can utilize, for example, RT-qPCR of specific transcripts, such as IFN-gamma (IFNG), Perforin 1 (PRF1) and Granzyme B (GZMB) after T-cell staining and isolation.

By qRT-PCR, gluten challenge increased multiple mRNAs over the CLIP control, including IL2, IL21, IL10, IL25 in CD4⁺ T cells, activation markers in CD8⁺ T cells (IFNG, PRF1, CD38, CD25) and proliferative markers in both (PCNA, CCND1).

A determination of a positive test for active celiac disease or a predisposition to celiac disease may be provided to a patient or a suitable medical practitioner.

### Screening Methods

In some aspects of the invention, methods and culture systems are provided for screening candidate agents or cells for an activity of interest. In these methods, candidate agents or cells are screened for their effect on cells in the organoids of the invention. Organoids of interest include those comprising unmodified cells, and those comprising experimentally modified cells, and an agent may be tested prior to, or following a gluten challenge as described above. The hallmarks of a celiac response may be measured as described above with respect to disease hallmarks, where an agent that is useful in preventing or treating disease will reduce the number or level of one or more hallmarks relative to a positive control.

The effect of an agent or cells is determined by adding the agent or cells to the cells of the cultured explants as described herein, usually in conjunction with a control culture of cells lacking the agent or cells. The effect of the candidate agent or cell is then assessed by monitoring one or more output parameters. Parameters are quantifiable components of explants or the cells thereof, particularly components that can be accurately measured, in some instances in a high throughput system. For example, a parameter of the explant may be the growth, differentiation, gene expression, proteome, phenotype with respect to markers etc. of the explant or the cells thereof, e.g. any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

In some embodiments, candidate agent or cells are added to the cells within the intact organoid. In other embodiments, the organoids are dissociated, and candidate agent or cells is added to the dissociated cells. The cells may be freshly isolated, cultured, genetically altered as described above; or the like. The cells may be environmentally induced variants of clonal cultures: e.g. split into independent cultures and grown into organoids under distinct conditions, for example with or without pathogen; in the presence or absence of other cytokines or combinations thereof. The manner in which cells respond to an agent, particularly a pharmacologic agent, including the timing of responses, is an important reflection of the physiologic state of the cell.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA, antisense molecules, or miRNA, or nucleic acids that encode polypeptides. Many vectors useful for transferring nucleic acids into target cells are available. The vectors may be maintained episomally, e.g. as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc. Vectors may be provided directly to the subject cells. In other words, the pluripotent cells are contacted with vectors comprising the nucleic acid of interest such that the vectors are taken up by the cells.

Methods for contacting cells with nucleic acid vectors, such as electroporation, calcium chloride transfection, and lipofection, are well known in the art. Alternatively, the nucleic acid of interest may be provided to the subject cells via a virus. In other words, the pluripotent cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the nucleic acid encoding the reprogramming factors into packaging cell lines and of collecting the viral particles that are generated by the packaging lines are well known in the art.

Vectors used for providing nucleic acid of interest to the subject cells will typically comprise suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing reprogramming factors to the subject cells may include genes that must later be removed, e.g. using a recombinase system such as Cre/Lox, or the cells that express them destroyed, e.g. by including genes that allow selective toxicity such as herpesvirus TK, bcl-xs, etc

Candidate agents of interest for screening also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

If the candidate polypeptide agent is being assayed for its ability to inhibit aggregation signaling intracellularly, the polypeptide may comprise the polypeptide sequences of interest fused to a polypeptide permeant domain. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide comprises the HIV-1 that basic region amino acid sequence, which may include, for example, amino acids 49-57 of naturally-occurring that protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

If the candidate polypeptide agent is being assayed for its ability to inhibit aggregation signaling extracellularly, the polypeptide may be formulated for improved stability. For example, the peptides may be PEGylated, where the polyethyleneoxy group provides for enhanced lifetime in the blood stream. The polypeptide may be fused to another polypeptide to provide for added functionality, e.g. to increase the in vivo stability. Generally such fusion partners are a stable plasma protein, which may, for example, extend the in vivo plasma half-life of the polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they comprise, or they may contain more than one polypeptide agent.

The candidate polypeptide agent may be produced from eukaryotic produced by prokaryotic cells, it may be further processed by unfolding, e.g. heat denaturation, DTT reduction, etc. and may be further refolded, using methods known in the art. Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine. The polypeptides may have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The candidate polypeptide agent may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Alternatively, the candidate polypeptide agent may be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In some cases, the candidate polypeptide agents to be screened are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of explant or cell samples, usually in conjunction with explants not contacted with the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow-through method. Alternatively, the agents can be injected into the explant, e.g. into the lumen of the explant, and their effect compared to injection of controls.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the growth rate.

Screens for agents to prevent or treat disease. Other examples of screening methods of interest include methods of screening a candidate agent for an activity in treating or preventing a disease. In such embodiments, the explant models the disease, e.g. the explant may have been obtained from a diseased tissue, or may be experimentally modified to model the disease by, e.g., genetic mutation. Parameters such as explant growth, cell viability, cell ultrastructure, tissue ultrastructure, etc. find particular use as output parameters in such screens.

Screens to determine the pharmacokinetics and pharmacodynamics of agents. Other examples include methods of screening a candidate agent for toxicity to tissue. In these applications, the cultured explant is exposed to the candidate agent or the vehicle and its growth and viability is assessed. In these applications, analysis of the ultrastructure of the explants is also useful.

### High Throughput Screens

In some aspects of the invention, methods and culture systems are provided for screening candidate agents in a high-throughput format. By “high-throughput” or “HT”, it is meant the screening of large numbers of candidate agents or candidate cells simultaneously for an activity of interest. By large numbers, it is meant screening 20 more or candidates at a time, e.g. 40 or more candidates, e.g. 100 or more candidates, 200 or more candidates, 500 or more candidates, or 1000 candidates or more.

In some embodiments, the high throughput screen will be formatted based upon the numbers of wells of the tissue culture plates used, e.g. a 24-well format, in which 24 candidate agents (or less, plus controls) are assayed; a 48-well format, in which 48 candidate agents (or less, plus controls) are assayed; a 96-well format, in which 96 candidate agents (or less, plus controls) are assayed; a 384-well format, in which 384 candidate agents (or less, plus controls) are assayed; a 1536-well format, in which 1536 candidate agents (or less, plus controls) are assayed; or a 3456-well format, in which 3456 candidate agents (or less, plus controls) are assayed. High throughput screens formatted in this way may be achieved by using, for example, transwell inserts. Transwell inserts are wells with permeable supports, e.g. microporous membranes, that are designed to fit inside the wells of a multi-well tissue culture dish. In some instances, the transwells are used individual. In some instances, the transwells are mounted in special holders to allow for automation and ease of handling of multiple transwells at one time.

To achieve the numbers of organoids necessary to perform a high-throughput screen, a primary organoid (that is, an organoid that has been cultured directly from tissue fragments) is dissociated into a single cell suspension and replated across multiple transwells to generate secondary organoids in a multiwell format. Dissociation may be by any convenient method, e.g. manual treatment (trituration), or chemical or enzymatic treatment with, e.g. EDTA, trypsin, papain, etc. that promotes dissociation of cells in tissue. The dissociated organoid cells are then replated in transwells at a density of 10,000 or more cells per 96-well transwell, e.g. 20,000 cells or more, 30,000 cells or more, 40,000 cells or more, or 50,000 cells or more. Additional iterations of dissociation and plating may be performed to achieve the desired numbers samples of organoids to be treated with agent.

In some embodiments, the secondary (or tertiary, etc.) organoids may be cultured first, after which candidate agents or cells are added to the organoid cultures and parameters reflective if a desired activity are assessed. In other embodiments, the candidate agents or cells are added to the dissociated cells at replating. This latter paradigm may be particularly useful for example for assessing candidate agents/cells for an activity that impacts the differentiation of cells of the developing organoid. Any one or more of these steps may be automated as convenient, e.g. robotic liquid handling for the plating of explants, addition of medium, and/or addition of candidate agents; robotic detection of parameters and data acquisition; etc.

### Utility

Organoids prepared by the subject methods may be used in basic research, e.g. to better understand the basis of disease, and in drug discovery, e.g. as reagents in screens such as those described further below, and for diagnostic purposes. Organoids are also useful for assessing the pharmacokinetics and pharmacodynamics of an agent, e.g. the ability of a mammalian tissue to absorb an active agent, the cytotoxicity of agents on primary mammalian tissue or on oncogenic mammalian tissue, etc.

The following examples are put forth so as to provide those of ordinary skill in the art with a complete disclosure and description of how to make and use the subject invention, and are not intended to limit the scope of what is regarded as the invention.

### Experimental

Celiac disease (CeD) is a prevalent and potentially disabling condition in which dietary exposure to gluten induces autoimmune destruction of intestinal epithelium with associated symptomatology. The pathogenesis of CeD is presumed to initiate with gluten-dependent T cell activation as inferred by demonstration of gliadin-reactive T cell receptors (TCRs) and HLA-DQ2 and -DQ8 as major risk factor alleles. Despite substantial insights into CeD pathophysiology to date, investigations have been substantially hampered by lack of in vivo and in vitro experimental models. In particular, in vitro studies of CeD have suffered from a singular lack of a holistic tissue culture model that preserves intestinal epithelium en bloc with endogenous diverse infiltrating immune populations without reconstitution. Although conventional organoid models reliably propagate intestinal epithelium from CeD patients, immune components are notably absent.

We have developed novel air-liquid interface (ALI) organoid models propagating intestinal epithelial cells or cancer cells alongside endogenous stroma including fibroblasts and immune cells. We have extended the ALI method to robust organoid culture of endoscopic biopsies from CeD patients in which intestinal epithelium is co-preserved with T and B cells, myeloid cells and fibroblasts without reconstitution. Notably, in vitro addition of gliadin to ALI CeD organoids induces epithelial death and hyperproliferation, histologic hallmarks of CeD. Crucially, gliadin treatment of ALI organoids rapidly stimulates T cell activation and expansion in organoids from CeD patients, but not from non-CeD controls. Further, single cell sequencing readily demonstrates known gliadin-binding TCR clonotypes within organoid-resident T cells.

The holistic ALI celiac organoid method is used to gain previously inaccessible insights into the immediate-early events following gliadin exposure, focusing on immune-epithelial crosstalk. CeD organoids can be used to functionally deconstruct the essential roles of immune cell types and cytokines during the gluten-induced autoimmunity by systematic pharmacologic modulation, again with single cell measurement of immune and epithelial perturbations. Overall, we capitalize on a novel organoid methodology preserving both the epithelial and immune components of CeD to dissect pathways of gluten-induced autoimmunity, with both basic and translational implications.

Celiac disease (CeD) is a common and potentially disabling autoimmune disorder where dietary gluten and MHC class II risk alleles, HLA-DQ2 or HLA-DQ8, initiate CD4+ T cell-dependent small intestinal mucosal injury. CeD diagnosis relies on serum antibody detection and confirmatory endoscopy, or oral symptomatic gluten challenge, with shortcomings of sensitivity, specificity and patient discomfort. Numerous questions regarding CeD pathogenesis abound, including the nature of inflammatory crosstalk between epithelium and immune cells, the definition of cellular immune cascades, relative contributions of intraepithelial versus lymphoid/peripheral blood T cells, and identity of essential gluten-presenting cells. Such investigations have suffered from a singular lack of a robust CeD tissue culture system integrating human intestinal epithelium with stroma and endogenous intraepithelial immune components without artificial reconstitution.

Healthy human small intestine tissue from biopsies done in 2 gastroenterology clinics were used, as well as hospital-provided tissue from resections from Whipple procedures, peripheral intestinal tissue from tumor surgeries and tissue from short bowel syndrome patients. These specimens, containing epithelial cells and stromal cells, are processed by mechanical mincing and grown in an air-liquid interface within a collagen gel, within a trans-well (“inner dish”) in which direct air exposure is obtained from the top; whilst contact with tissue culture media contained in an “outer dish” is obtained from the bottom via the trans-well permeable membrane, generating an Air-Liquid Interface (ALI), shown in FIG. 1.

Human organoids—4-8 biopsy bites are mechanically minced with scissors into microscopic fragments that are grown from 7-12 days en bloc as 3D organoids that contain epithelium, mesenchymal stroma, and, importantly, a diverse and functional intestinal immune system (FIG. 1A-B).

Organoids were treated with deamidated gliadin peptides as a well-established mimic of the highly immunogenic gluten protein component of common dietary starches. Such deamidated gluten peptides are widely used in the celiac field as they are well established to bind HLA-DQ2 and stimulate CeD-specific CD4⁺ T cells. As a control, ALI organoids were treated with a CLIP peptide derived from the MHC invariant chain which is competent to be presented by all MHC class II molecules. The gliadin peptides, or the CLIP peptides, were added to a final concentration in media of 10 μM. Specifically, deamidated gliadin may be 50:50 mixture of gliadin-α1 (5 μM, 14 aa, Genscript) and gliadin-α2 (5 μM, 13 aa, Genscript) or CLIP control peptide (10 μM, Genscript).The organoids are harvested from the culture 2 days later for analysis (FIG. 1C).

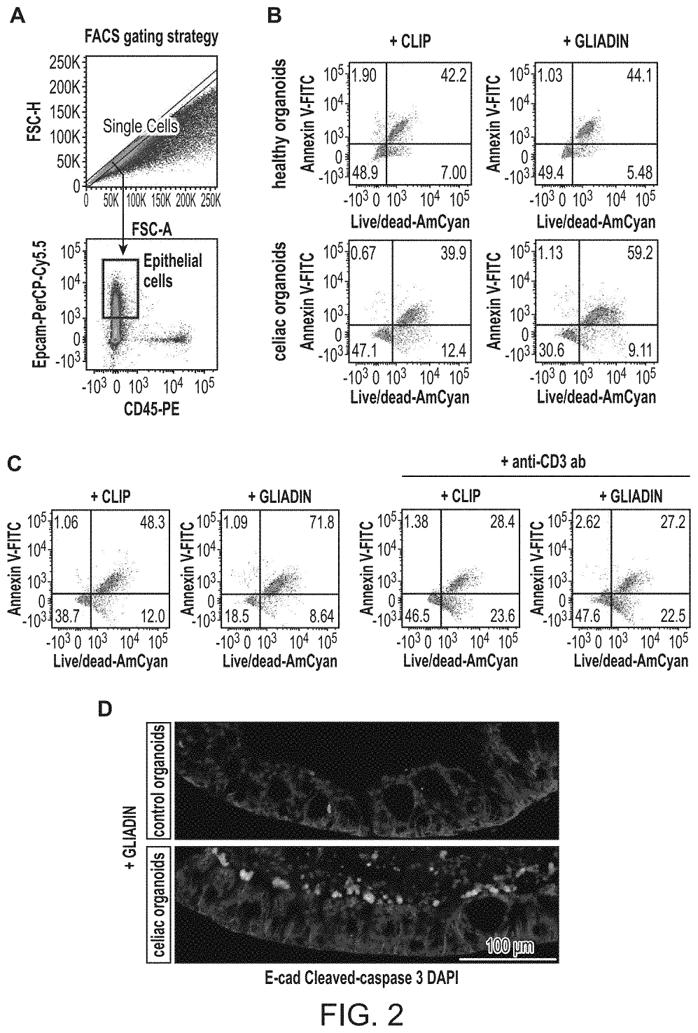

Shown in FIG. 2, single cells harvested from the organoids were sorted and analyzed after immuno-staining for the epithelial cell marker EpCam and the apoptosis marker Annexin V together with a dead cell marker, showing that treatment with gliadin but not CLIP leads to an increase in cell death. The data confirmed an increase in epithelial cell death in celiac-organoids treated with gliadin but no effects in healthy-organoids (FIG. 2A-B).

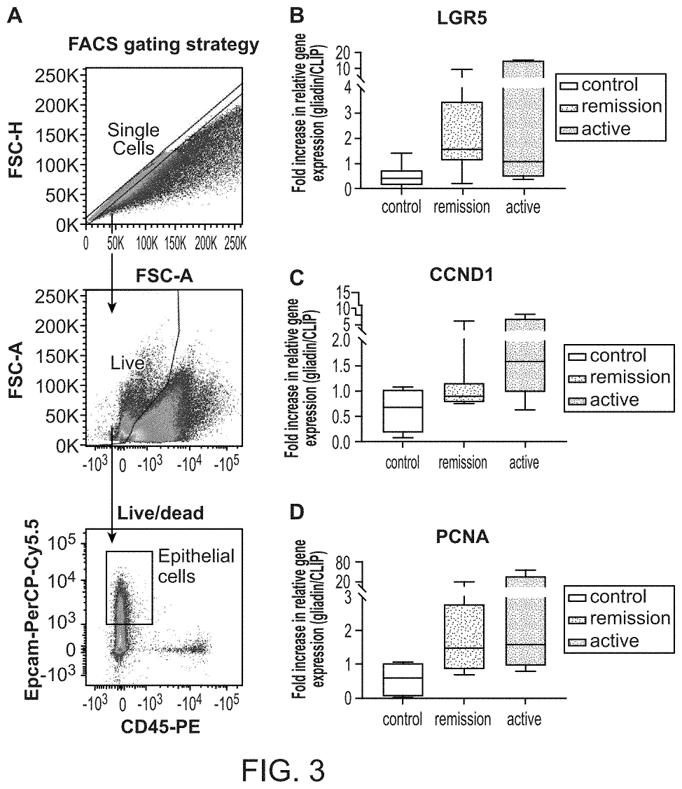

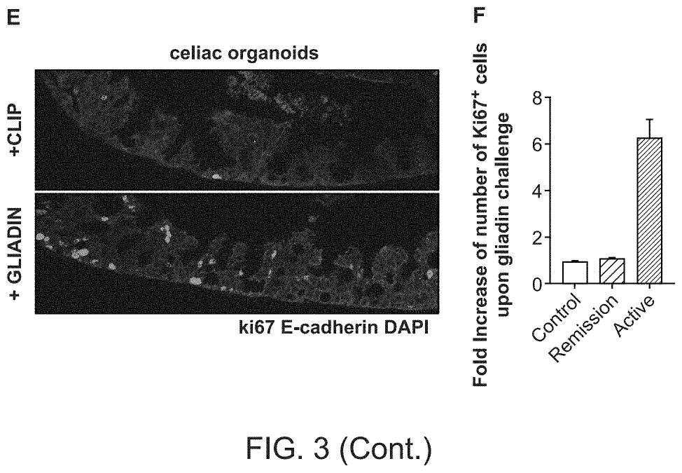

Treating intact active CeD organoids with the validated anti-human CD3 antibody OKT3 efficiently depletes ˜90% of CD4+ and ˜80% of CD8+ T cells and abrogates gliadin-induced epithelial apoptosis, strongly indicating T cell dependency (FIG. 3C). Anti-CD3 also decreased baseline apoptosis, suggesting basal T cell-epithelial crosstalk.

Epithelial cell-death can also be seen after positive immunostaining signal of the apoptosis marker Cleaved-Caspase 3 by IF in celiac organoids after gliadin-challenge but not CLIP (FIG. 2C). FIG. 3 reveals increase in stem and proliferative markers such as LGRS, CCND1 and PCNA by RT-qPCR of FACS-sorted EpCAM+ epithelial cells (FIG. 3A-D) and reveals a 6× fold increase in Ki67+ proliferative cells in organoids-derived from active celiac patients by IF staining (FIG. 3E-F).

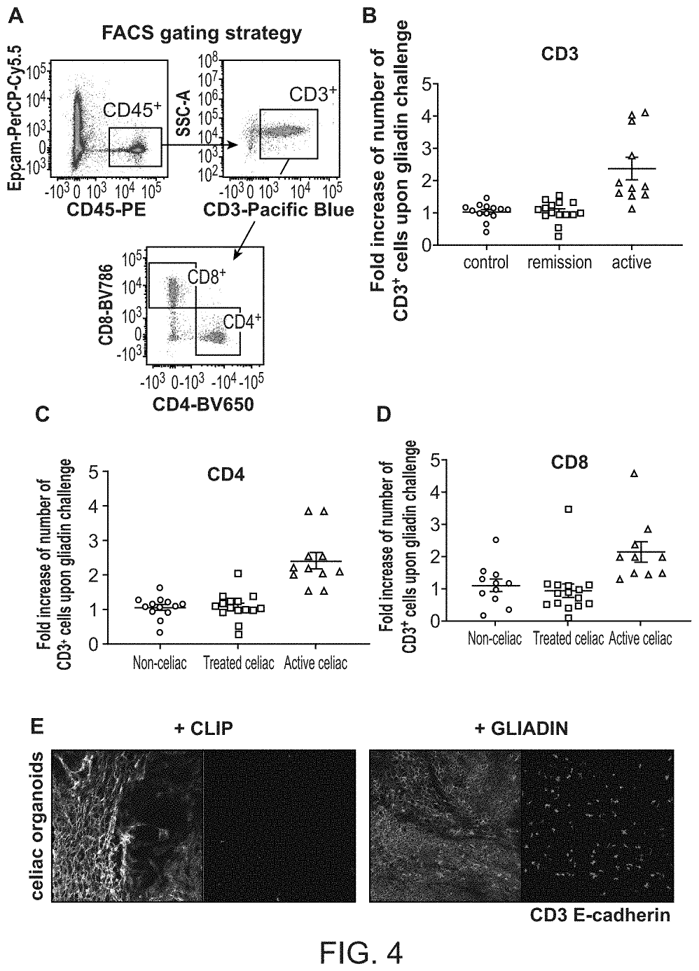

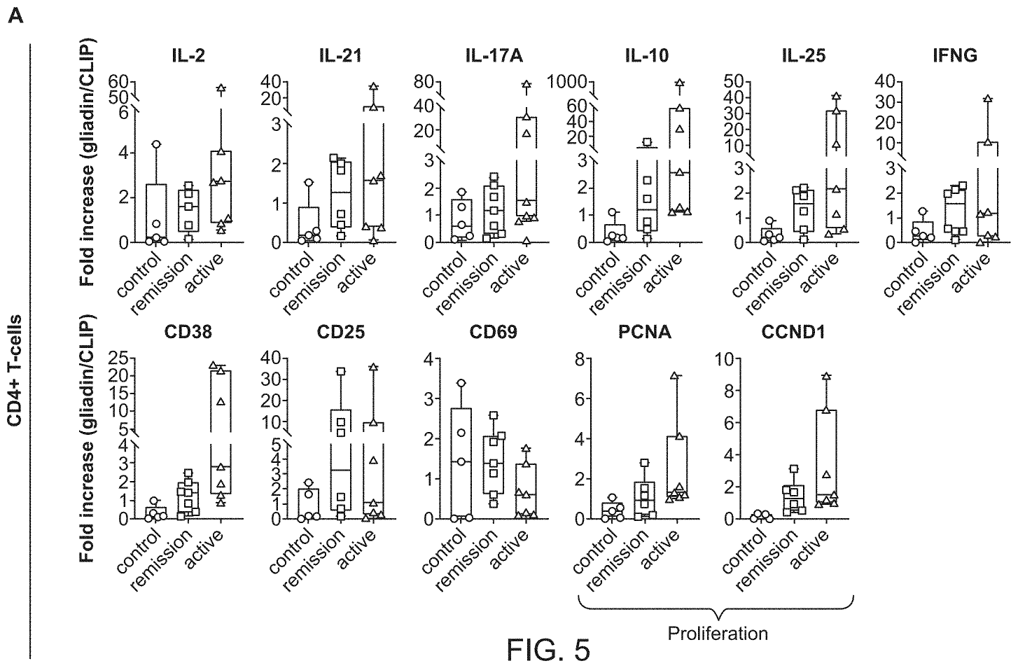

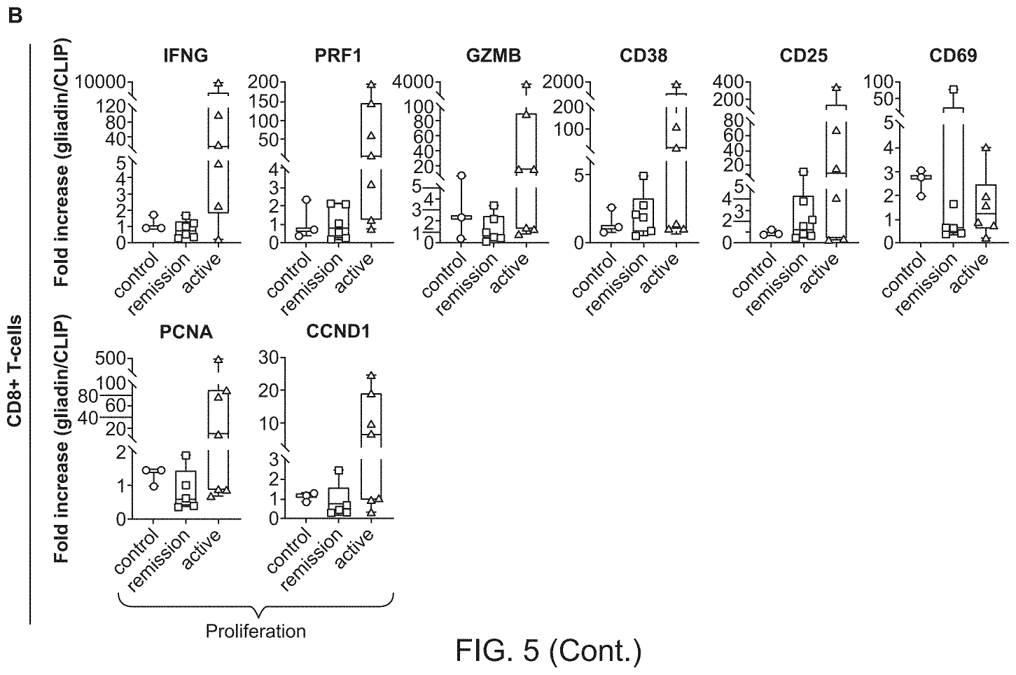

In FIG. 4 we confirmed the expansion of T-cells observed in celiac-organoids treated with gliadin but no changes when celiac-organoids were treated with CLIP by FACS (FIG. 4A-D) and by IF (FIG. 4E). In FIG. 5 we show that T cells FACS-sorted from the culture show an increase in transcripts upon gliadin treatment versus CLIP treatment of several pro-inflammatory cytokines such as IL-2, IL-22, IL-17A, IL-10, IL-25 and IFNG; of CD38, a memory T-cell marker and CD25, the IL-2 receptor; and of proliferation markers such as CCND1 and PCNA in organoids derived from active celiac patients and to some extent in organoids derived from remission celiac patients, but not in non-celiac healthy-derived organoids (FIG. 5A-B).

We provide application of a novel human air-liquid interface (ALI) organoid model of celiac disease (CeD), preserving intestinal epithelium with endogenous infiltrating immune cells en bloc without reconstitution, to the study of CeD pathogenesis. An immune response to ingested gluten peptides causes small intestinal mucosal destruction in a subset of individuals carrying MHC class II alleles HLA-DQ2 or -DQ8. Following wheat ingestion, gliadin peptides within gluten are deaminated by tissue transglutaminase 2 (TG2) in the lamina propria. Deamination introduces negative charges which enhance MHC binding, culminating in a gliadin-specific TH1 mediated HLA-DQ2- or DQ8-restricted immune response with T cell-mediated intestinal inflammation and mucosal injury and anti-gliadin and anti-TG2 autoantibodies. Concurrently, gluten-specific CD4⁺ and disease-associated CD8⁺ and γδ T cells are elevated in intestinal epithelium and blood. Characteristic histology includes villous atrophy, crypt hyperproliferation, lamina propria inflammation and intraepithelial lymphocytosis alongside malabsorption and gastrointestinal and extraintestinal symptoms.

While CeD-associated MHC class II is the strongest risk factor, and T cell-mediated cytotoxicity is crucial, other factors including dysregulated immunity and environmental triggers likely contribute. In a healthy population, 30-35% carry HLA-DQ2 or HLA-DQ8, but only 3-5% develop CeD. GWAS studies identified 40 non-HLA CeD loci, some implicating innate immunity or barrier function. Epithelial barrier dysfunction is a CeD hallmark but could either be directly causal or a consequence of inflammation. Innate immunity may induce mucosal injury/barrier dysfunction and subsequent TG2 activation to initiate CeD. Improved disease models are needed to dissect epithelial and immune cell interactions in CeD, as extensively explored herein using organoids to define the acute time course of gluten-induced immune-epithelial crosstalk, and to provide diagnostic methods with these cultures.

The CeD organoid model from human endoscopic biopsies preserves both the syngeneic intestinal epithelium and native intestinal immune cells without reconstitution, and importantly exhibits T cell activation in response to in vitro gluten challenge. This is the first native organoid model of CeD. Air-liquid interface (ALI) organoids have both epithelial and stromal components from diverse mouse and human organs and tumors (Nature Medicine 2009, Nature Medicine 2014, Cell 2018). The ALI method is a truly organotypic approach that cultures larger fragments of tissue than does submerged Matrigel methods and thus allows larger sheets of cells to preservation epithelium and stroma together as a cohesive unit en bloc. In ALI, adequate oxygenation of the large tissue fragments is achieved by culture within a transwell (“inner dish”) containing extracellular matrix and cells in which direct air exposure is obtained on top; tissue culture media is exclusively contained in the “outer dish” and enters the inner dish via the transwell permeable membrane. Using duodenal endoscopic biopsies from CeD patients and normal (i.e. non-CeD) controls, we optimized human ALI organoid culture. By improved specimen processing and Wnt/EGF/Noggin/R-spondin (WENR) media, with >95% success rate and robust continuous culture for >100 days (longest times attempted). The ALI organoids recapitulate villus- and crypt-like domains with basally-located proliferation.

The ALI organoids preserve numerous stromal populations. To emphasize, the stromal cells are not added exogenously by reconstitution but are rather retained endogenously en bloc along with the epithelium, with distinct SMA⁺ and PDGFRα⁺ myofibroblasts and PGP9.5⁺ neural cells present. Crucially, ALI organoids also robustly recapitulate the epithelial lymphocyte infiltration of CeD. ALI organoids from endoscopic duodenal biopsies of active CeD patients robustly preserve endogenous T, B cells and macrophages en bloc with the intestinal epithelium without reconstitution. These immune populations persist for at least 3 weeks.

CeD is characterized by seminal histologic characteristics, including the well-known villus blunting, resulting from immune-mediated epithelial cell death. Less appreciated in CeD is the uniform and simultaneous presence of crypt hypertrophy, which may represent compensatory proliferation. We examined the ability of CeD ALI organoids that widely preserve diverse immune populations to recapitulate gluten induction of both epithelial cell death and secondary epithelial proliferation. Active CeD cultures were treated with deamidated gliadin peptides as a well-established mimic of the highly immunogenic gluten component of common dietary starches (see Sjostrom, H., et al. Identification of a gliadin T-cell epitope in coeliac disease: general importance of gliadin deamidation for intestinal T-cell recognition. Scand J Immunol 48, 111-115 (1998), herein specifically incorporated by reference). Such deamidated gluten peptides are widely used in the celiac field as they are well established to bind HLA-DQ2 and stimulate CeD-specific CD4⁺ T cells. As a control, ALI organoids were treated with a CLIP peptide derived from the MHC invariant chain which is competent to be presented by all MHC class II molecules. Under these conditions, gliadin, but not CLIP, induced epithelial cell death in ALI organoids from patients with active CeD but not in control organoids from non-CeD patients. Moreover, gliadin but not CLIP increased proliferative markers, again in active CeD organoids but not controls (FIG. 5C,D). In proliferation studies, organoids were switched to differentiation media lacking Wnt/R-spondin to induce a quiescent baseline. Thus, ALI organoids recapitulate canonical CeD gluten-dependent epithelial apoptosis and proliferation.

We further characterized gliadin-induced T cell responses in ALI CeD organoids. Pre-established CeD organoids were treated with deamidated gliadin or CLIP in vitro. CD4 and CD8 IF revealed that gliadin but not CLIP induced hotspot foci of T cells in organoids. FACS confirmed gliadin stimulation of CD3⁺, CD4⁺ and CD8⁺ T cell abundance in organoids vs. CLIP, but CD19⁺ B cells were unaltered. Notably, elevated gliadin:CLIP ratios of CD3⁺, CD4⁺ and CD8⁺ abundance were highly specific for active CeD organoids and not for organoids from control/non-CeD or remission celiac (i.e., asymptomatic CeD patients on a GFD).

Gliadin also activated T cells within CeD organoids by multiple criteria. We treated ALI organoids from (1) active CeD, (2) remission CeD or (3) non-CeD endoscopic biopsies with either deamidated gliadin peptide or CLIP, followed by FACS purification of CD4⁺ or CD8+ T cells. By qRT-PCR, gliadin increased multiple mRNAs over the CLIP control, including IL2, IL21, IL10, IL25 in CD4⁺ T cells, activation markers in CD8+ T cells (IFNG, PRF1, CD38, CD25) and proliferative markers in both (PCNA, CCND1). Similarly, scRNA-seq revealed that gliadin but not CLIP induced cytotoxic markers (IFNG and PRF1 and to a lesser extent GZMB and IL2) selectively in T cell subsets but not in other hematopoietic cells. Tandem single cell TCR-seq of T cell receptor (TCR) CDR3 regions from gliadin-treated CeD organoids showed TCR sequence matched a gliadin-binding TCR clonotype from the Koning/Leiden database, reaffirming the validity of CeD ALI organoids. Further, bioinformatic GLIPH analysis binned additional TCR clonotypes having sequence-predicted antigenic overlap with known Koning/Leiden gliadin-binding TCRs.

Overall, we have established a holistic organoid system allowing en bloc preservation and expansion of CeD endoscopic biopsies as intestinal epithelium alongside diverse immune cells (T cell subsets, B, plasma cells, myeloid), notably without reconstitution. We exploit this unique methodology to obtain molecular and cellular insights into the immediate-early sequence of events following antigenic gluten exposure within the intestinal epithelium.

CeD ALI organoids are ideally suited for the longitudinal sampling of a single biological sample after gliadin-exposure, which would otherwise require repeated endoscopy over hours to days after gluten ingestion. This can be determined by single cell RNA-seq (transcriptome), suspension-CyTOF (proteome) and imaging-CyTOF (spatial) analysis of the acute time course of gluten-stimulated immune events, constructing a multi-omic single cell network model of the immune cascades and epithelial crosstalk in CeD. Additionally, the tractable and holistic CeD organoids allow the first in vitro deconstruction of essential CeD immune cell types and cytokines in a human experimental model.

The pronounced specificity of gliadin responses for ALI organoids from CeD patients but not from non-CeD controls provides utility as an in vitro CeD diagnostic assay using a biological readout from living cells.

CeD ALI organoids are ideally suited for the longitudinal sampling of a single biological sample after gliadin exposure, which would otherwise require repeated endoscopy of an identical area of mucosa over hours to days after gluten ingestion. Here, we exploit the CeD organoid system to generate the first single cell landscape of acute gliadin-induced immune responses and epithelial crosstalk in CeD over time, integrating transcriptomic, proteomic and spatial single cell technologies.

Organoids from CeD and not control patients strongly respond to in vitro stimulation of gliadin, with disease hallmarks including epithelial apoptosis, reactive hyperproliferation, and crucially T cell activation and expansion alongside known gliadin-binding TCR clonotypes. The TCR clonotype sequence unique to every T cell can be exploited as a barcode' to identify which T cells have clonally expanded, suggesting potential involvement in recognition of gluten. We grew ALI organoids from endoscopic duodenal biopsies from a single active celiac patient and performed tandem TCR-seq/5′RNA-seq on FACS-purified CD45⁺ immune cells with or without gliadin stimulation. This revealed preservation of a highly diverse intestinal immune compartment, consisting of plasma B cells, mature B cells, various T cell subsets (memory CD4, memory CD8, replicating T cells, FOXP3⁺ T_{reg}), myeloid, and basophil-like cells. Moreover, TCR sequence homologies to known gluten-reactive T cell clones were readily identified.

Each ALI dish typically yields ˜1-2×10⁷ live cells/dish. The (1) CD45⁺ immune (˜15% of total) and the (2) EPCAM⁺ epithelial (majority) compartments are FACS-purified from each condition followed by single cell 5′ RNA/TCR-seq with the 10× Genomics Chromium Immune Profiling system and NextSeq/HiSeq sequencing as we described. The resultant single cell 5′ transcriptome and TCRαβ CDR3 sequences are analyzed to determine gliadin-induced expression profiles of (1) CeD-associated immune cell populations and (2) intestinal epithelium with particular interest in T cells having TCRs with known or GLIPH-binned gliadin specificities. This is also be analyzed by the pseudotime algorithm to cluster genes by expression along putative differentiation trajectories mapping gliadin-induced transitions. Epithelial cell death and proliferation is measured in EPCAM+ cells by Annexin V/7-AAD FACS and expression of proliferative stem cell markers (PCNA, MKI67, CCND1, LGR5). Further analysis is provided by overlay with DQ2:gliadin tetramers that directly identify gluten-reactive T cells. These analyses capture discrete kinetics of the gliadin-induced immune response with accompanying changes in the intestinal epithelium. The inclusion of gliadin-specific tetramers will detect gluten-specific T cells and determine their transcriptome and TCR repertoire at single cell resolution. In parallel, the TCR sequencing will link the TCR repertoire of the disease-associated T cell populations to their phenotypes and differentiation stages as assessed at the RNA level.

The same CD45⁺ CeD organoid time course (0, 6 h, 1 d, 2 d) +/− gliadin and fresh biopsy (n=10 CeD and controls) isanalyzed with 40-plex CyTOF immune antibody panel, including antibodies for gliadin-regulated candidates. The generated CyTOF data is analyzed by the HSNE algorithm for unbiased exploration of millions of cells at single cell resolution without the need for data downsampling.

Our initial results demonstrated clear cellular organization in the intestinal organoids by IF staining, with CD3⁺ T cells embedded within the EPCAM⁺ epithelial cell layer (intraepithelial lymphocytes, IELs) and CD3⁺ T cells, CD14⁺ myeloid cells and CD19⁺ B cells in the underlying organoid lamina propria. Also, gliadin but not CLIP induced foci of CD4⁺ and CD8⁺ T cells within CeD organoids. To comprehensively visualize tissue architecture and high-dimensional cellular landscape of the organoids in situ, imaging-CyTOF for multiplexing of up to 40 markers at subcellular resolution is utilized. This combines IHC staining with metal isotope-conjugated antibodies, laser ablation and mass spectrometry-based detection to produce high-content images. The generated imaging-CyTOF data is analyzed in a data-driven fashion by (1) MCD Viewer (Fluidigm) for marker visualizations and (2) ImaCytE software. This computational pipeline allows unbiased high-dimensional analysis of cell interactions from cell-segmented images, revealing cellular microenvironments in the organoids in situ.

Treating intact active CeD organoids with the validated anti-human CD3 antibody OKT3 efficiently depletes ˜90% of CD4⁺ and ˜80% of CD8⁺ T cells and abrogates gliadin-induced epithelial apoptosis, strongly indicating T cell dependency. Anti-CD3 also decreased baseline apoptosis, suggesting basal T cell-epithelial crosstalk. Sequelae of T cell depletion in CeD organoids is assessed as follows. Intact pre-established active CeD organoids undergo anti-CD3/OKT3 pan-T cell depletion with 48 h anti-CD3 pretreatment and then anti-CD3+/− gliadin or CLIP for 48 h. Endpoints include (1) quantitation of EPCAM⁺ epithelial cell death by Annexin V/AmCyan FACS and cleaved caspase-3 IF, (2) scRNA-seq and FACS of immune and intestinal epithelial cells per Aim 1A (n=3 patients) and (3) Luminex and Nanostring quantitation of secreted cytokines and bulk digital transcript immune profiling (n=6 patients). These also detect B and myeloid cell abundance and activation.

Notably, gliadin strongly induces IL-15 expression in active CeD organoids. IL-15 loss-of-function is studied in gliadin-treated, active CeD organoids +/− IL-15 neutralization by soluble recombinant IL-15 receptor (soluble hIL15Rα, R&D #7194-IR) or anti-human IL-15 neutralizing antibody (R&D #MAB647). Endpoints include inhibition of the following gliadin-induced responses: (1) HLA-DQ2:gliadin tetramer-positive T cell abundance by FACS, (2) tetramer(+) CD4⁺ T cell or tetramer(−) CD8⁺ IEL activation by scRNA-seq and Nanostring digital transcript counting of FACS-sorted immune subsets and (3) inhibition of gliadin-induced epithelial apoptosis and proliferation. Gain-of-function recombinant IL-15 treatment (R&D #247-ILB) of CeD organoids enhances gliadin-induced (1) expansion of HLA-DQ2:gliadin tetramer⁸²-positive T cells by FACS, (2) activation of these tetramer(+) CD4⁺ T cells or tetramer(−) CD8⁺ IELs by scRNA-seq and FACS/Nanostring analysis of immune subsets and (3) induction of epithelial apoptosis and proliferation.

1. A method for culture of a mammalian organoid model for celiac disease, the method comprising:
culturing mammalian small intestine tissue comprising syngeneic intestinal epithelium and native intestinal immune cells, in a gel with an air-liquid interface, in a medium that supports maintenance and activity of both epithelial and immune cells for a period of at least 5 days.

2. The method of claim 1, further comprising the step of adding to the culture medium a dose of gluten-derived peptides in a dose effective to activate immune cells present in the culture.

3. The method of claim 2, wherein the gluten-derived peptides are peptides of wheat gliadin from about 8 to about 35 amino acids in length.

4. The method of claim 2, wherein the gliadin peptides are deamidated.

5. The method of claim 2, wherein the dose effective to activate immune cells present in the culture is a concentration of from 0.5 μM to 100 μM.

6. The method of claim 1, wherein the small intestine tissue is an endoscopic biopsy sample.

7. The method of claim 1, wherein the small intestine tissue is obtained from an individual suspected or known to have celiac disease.

8. The method of claim 1, wherein the medium comprises one or more of R-spondin, a WNT agonist, noggin, and EGF.

9. The method of claim 1, further comprising a step after gluten challenge of determining the presence of a hallmark of celiac disease.

10. The method of claim 9 wherein the hallmark of celiac disease is one or more of gliadin-presentation to T cells; T cell expansion; T cell activation; epithelial-cell death; and
increased epithelial cell proliferation.

11. The method of claim 10, wherein epithelial cell is measured by cell staining or RT-qPCR for apoptotic markers, including one or more of Annexin V, cleaved-caspase 3 and quantitating the level of relative to a control.

12. The method of claim 10, wherein epithelial cell proliferation is measured by cell staining or RT-qPCR for proliferation markers, including one or more of Ki67, CCND1, PCNA and quantitating the level of relative to a control.

13. The method of claim 10, wherein T cell proliferation is measured by quantitation of an increase in one or more of CD3⁺, CD4⁺ and CD8⁺ T cells in the culture relative to a control.

14. The method of claim 10, wherein T cell activation is measured by determining expression of one or more of IFN-gamma (IFNG), Perforin 1 (PRF1) Granzyme B (GZMB), IL2, IL21, ID10, IL25, CD38, CD25 in T cells present in the culture, relative to a control.

15. An in vitro organoid culture derived by the method of claim 1.

16. A method for screening a candidate agent for an effect on a mammalian tissue, the method comprising:
contacting a candidate agent with an organoid culture according to claim 15, and determining the effect of the agent a hallmark of celiac disease.

17. The method of claim 16, wherein the determining step comprises the steps of claim 10.

18. A method for determining the presence of active celiac disease or a predisposition to celiac disease in an individual, the method comprising:
obtaining a small intestine tissue sample from the individual;
culturing the tissue in a method according to claim 2; and determining the presence of a hallmark of celiac disease, wherein the presence of the hallmark response compared to a control determines that an individual has celiac disease or is pre-disposed to celiac disease.
